(19) United States
(12) Patent Application Publication
(10) Pub. No.: US 2023/0329502 A1
(43) Pub. Date: Oct. 19, 2023

(54) VACUUM CLEANER ODOR DIFFUSION SYSTEM

(71) Applicant: SHARKNINJA OPERATING LLC, Needham, MA (US)

(72) Inventor: Brian CHIRIKJIAN, Cambridge, MA (US)

(21) Appl. No.: 18/213,129

(22) Filed: Jun. 22, 2023

Related U.S. Application Data
(63) Continuation-in-part of application No. 17/857,639, filed on Jul. 5, 2022.
Application No. 17/857,639 is a continuation-in-part of application No. 17/843,692, filed on Jun. 17, 2022.
(60) Provisional application No. 63/228,905, filed on Aug. 3, 2021.

(51) Int. Cl. A47L 9/28; A61L 9/12; A47L 7/00; A47L 5/30; A47L 7/04

(52) U.S. Cl. CPC A47L 9/2873; A61L 9/12; A47L 7/0061; A47L 5/30; A47L 7/04; A61L 2209/133; A47L 2201/024

(57) ABSTRACT

A cleaning system includes a docking station. The docking station includes a station suction inlet configured to be fluidly coupled to a vacuum cleaner, a station dust cup configured to be removably fluidly coupled to the docking station, where the station dust cup includes a debris cavity; an odor control assembly fluidly coupled to the station dust cup; and a station suction motor configured to cause air to flow into the station suction inlet and through the station dust cup. The station suction motor is configured to generate an airflow through the odor control assembly and into the debris cavity.

### CROSS-REFERENCE TO RELATED APPLICATIONS

The present application is a continuation-in-part of U.S. application Ser. No. 17/857,639, filed Jul. 5, 2022, and U.S. application Ser. No. 17/843,692, filed Jun. 17, 2022, which claims the benefit of U.S. Application Ser. No. 63/228,905, filed Aug. 3, 2021, the entire teachings of which are hereby incorporated herein by reference.

### TECHNICAL FIELD

The present disclosure is generally related to surface treatment devices and more specifically to vacuum cleaners configured to interface with a docking station.

### BACKGROUND INFORMATION

Surface treatment devices are configured to remove at least a portion of any debris that is deposited on a surface to be cleaned (e.g., a floor). For example, the surface treatment apparatus may be a vacuum cleaner that includes a suction motor, a suction inlet, and a dust cup. The suction motor is configured to cause air to flow through the suction inlet and into the dust cup. As air is drawn into the suction inlet at least a portion of any debris on the surface to be cleaned may become entrained within the air. At least a portion of the entrained debris may be deposited within the dust cup for later disposal by a user of the vacuum cleaner. Frequency of disposal may be based, at least in part, on a volume of the dust cup. Increased dust cup volumes may result in increased overall weight and/or size of the vacuum cleaner. While smaller dust cup volumes may reduce a weight and/or size of the vacuum cleaner, it may result in more frequent disposal of debris, which may expose the user more frequently to the disposed debris.

### BRIEF DESCRIPTION OF THE DRAWINGS

These and other features and advantages will be better understood by reading the following detailed description, taken together with the drawings, wherein:

FIG. 1 is a schematic example of a vacuum cleaner docked with a docking station, consistent with embodiments of the present disclosure.

FIG. 2 is a schematic example of the vacuum cleaner of FIG. 1 having a dust cup in a manual emptying configuration, consistent with embodiments of the present disclosure.

FIG. 3 is a schematic example of the vacuum cleaner of FIG. 1 having the dust cup in an automated emptying configuration, consistent with embodiments of the present disclosure.

FIG. 4 is a perspective view of a vacuum cleaner docked with a docking station, consistent with embodiments of the present disclosure.

FIG. 5 is a perspective view of the vacuum cleaner of FIG. 4 being undocked from the docking station of FIG. 4, while one or more accessories of the vacuum cleaner remain docked with the docking station, consistent with embodiments of the present disclosure.

FIG. 6 is a perspective view of the docking station of FIG. 4, consistent with embodiments of the present disclosure.

FIG. 6A is a magnified view of a portion of the docking station of FIG. 4 corresponding to region 6A of FIG. 6, consistent with embodiments of the present disclosure.

FIG. 7 is cross-sectional view of a receptacle of the docking station of FIG. 4 for receiving the vacuum cleaner of FIG. 4, consistent with embodiments of the present disclosure.

FIG. 8 is a perspective view of the vacuum cleaner of FIG. 4 having a dust cup outlet in a closed configuration, consistent with embodiments of the present disclosure.

FIG. 8A is a magnified view of a portion of the vacuum cleaner of FIG. 4 corresponding to region 8A of FIG. 8, consistent with embodiments of the present disclosure.

FIG. 9 is a perspective view of the vacuum cleaner of FIG. 4 having the dust cup outlet in an open configuration, consistent with embodiments of the present disclosure.

FIG. 10 is a cross-sectional view of the vacuum cleaner and the docking station of FIG. 4 taken along the line X-X of FIG. 4, consistent with embodiments of the present disclosure.

FIG. 11 is a schematic example of a vacuum cleaner docked with a docking station, consistent with embodiments of the present disclosure.

FIG. 12A is a perspective view of the docking station, with the vacuum cleaner undocked from the docking station, consistent with embodiments of the present disclosure.

FIG. 12B is a perspective view of the station dust cup, showing the odor control cavity with the odor control assembly removed.

FIG. 12C is a top view of the station dust cup, showing the odor control cavity with the odor control assembly removed.

FIG. 13A is a perspective view of an odor control assembly for the vacuum cleaner of FIG. 11, consistent with embodiments of the present disclosure.

FIG. 13B is an exploded view of the odor control assembly for the vacuum cleaner of FIG. 11, consistent with embodiments of the present disclosure.

FIG. 13C is a front view of an odor control assembly for the vacuum cleaner of FIG. 11, consistent with embodiments of the present disclosure.

FIG. 13D is a side view of an odor control assembly for the vacuum cleaner of FIG. 11, consistent with embodiments of the present disclosure.

FIG. 13E is a front view of a dial body for the vacuum cleaner of FIG. 11, consistent with embodiments of the present disclosure.

FIG. 13F is a perspective view of a puck cartridge for the vacuum cleaner of FIG. 11, consistent with embodiments of the present disclosure.

FIG. 13G is a front view of a scent puck for the vacuum cleaner of FIG. 11, consistent with embodiments of the present disclosure.

FIG. 13H is a bottom perspective view of the puck cartridge for an odor control assembly for the vacuum cleaner of FIG. 11, consistent with embodiments of the present disclosure.

FIG. 14A is a front cross-sectional view of an odor control assembly mounted in a station dust cup, showing the inlet air path, consistent with embodiments of the present disclosure.

FIG. 14B is a front perspective view of an odor control assembly mounted in a station dust cup in a docking station for the vacuum cleaner of FIG. 11, consistent with embodiments of the present disclosure.

FIG. 14C is a rear perspective view of an odor control assembly mounted in a station dust cup in a docking station for the vacuum cleaner of FIG. 11, consistent with embodiments of the present disclosure.

FIG. 15 is a top cross-sectional view of an odor control assembly mounted in a station dust cup, showing the inlet air path, consistent with embodiments of the present disclosure.

FIG. 16 is a front cross-sectional view of a station dust cup, showing the outlet air path, consistent with embodiments of the present disclosure.

FIG. 17 is a side view of the docking station for the vacuum cleaner, showing the outlet port for the station dust cup, consistent with embodiments of the present disclosure.

FIG. 18A is a perspective view of a station dust cup, showing an alternate bleed hole location for the inlet air for the odor control assembly, consistent with embodiments of the present disclosure.

FIG. 18B is a front cross-sectional view of a station dust cup, showing an alternate bleed hole location for the inlet air for the odor control assembly, consistent with embodiments of the present disclosure.

FIG. 19 is a perspective view of the docking station for the vacuum cleaner, showing an alternate location for the odor control assembly, consistent with embodiments of the present disclosure.

FIG. 20 shows a front view of a docking station for a robotic vacuum cleaner that incorporates an odor control assembly, consistent with embodiments of the present disclosure.

FIG. 21 shows a front cross-sectional view of an odor control assembly in a docking station for a robotic vacuum cleaner, consistent with embodiments of the present disclosure.

### DETAILED DESCRIPTION

The present disclosure is generally related to a vacuum cleaner and a docking station configured to interface with the vacuum cleaner. The vacuum cleaner includes a cleaner suction motor, a cleaner suction inlet, and a cleaner dust cup. The cleaner suction motor is fluidly coupled to the cleaner suction inlet and the cleaner dust cup such that cleaner suction motor, when activated, draws air through cleaner suction inlet and into the cleaner dust cup. Air drawn through the cleaner suction inlet may have debris entrained therein. At least a portion of the entrained debris is deposited within the cleaner dust cup for later disposal. The cleaner dust cup can include a first emptying configuration and a second emptying configuration for removing debris from the cleaner dust cup. The first emptying configuration can correspond to a manual emptying configuration (e.g., for emptying the cleaner dust cup into a trash receptacle by a user) and the second emptying configuration can correspond to an automated emptying configuration (e.g., for emptying the cleaner dust cup using the docking station).

The docking station includes a station suction motor, a receptacle having a station suction inlet, and a station dust cup. The station suction motor is configured to cause air to flow into the station suction inlet and through the station dust cup. The receptacle is configured to interface with the vacuum cleaner such that vacuum cleaner removably couples to (docks with) the docking station. The cleaner dust cup can be transitioned to the automated emptying configuration when the vacuum cleaner is docked to the docking station and the station suction motor is activated. When in the automated emptying configuration, the cleaner dust cup and the station dust cup are fluidly coupled such that, when the station suction motor is activated, at least a portion of any debris stored within the cleaner dust cup is transferred into the station dust cup.

Use of the docking station to empty the cleaner dust cup may reduce a number times a user is exposed to debris collected by the vacuum cleaner (e.g., as a result of debris pluming during emptying). For example, the station dust cup may be configured to have a volume that is greater than the cleaner dust cup (e.g., a volume that is at least two times greater). As such, a user may dispose of collected debris less frequently, reducing exposure of the user to debris.

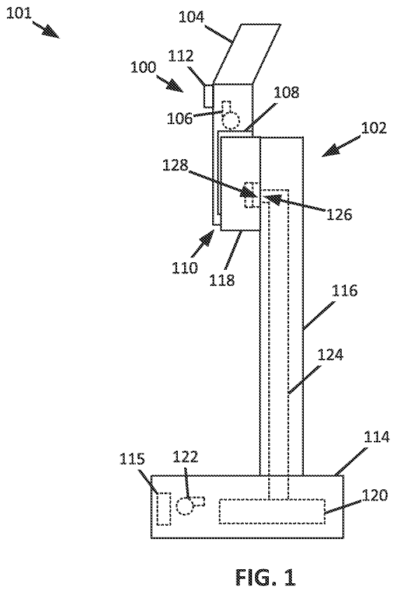

FIG. 1 shows a schematic example of a cleaning system 101 having a vacuum cleaner 100 removably coupled (docked) to a docking station 102. The vacuum cleaner 100 includes a handle 104, a cleaner suction motor 106, a cleaner dust cup 108, and a cleaner inlet 110. The cleaner suction motor 106 is fluidly coupled to the cleaner inlet 110 and the cleaner dust cup 108 such that, when the cleaner suction motor 106 is activated, air is caused to flow through the cleaner inlet 110 and into the cleaner dust cup 108. Air flowing through the cleaner inlet 110 may have debris entrained therein. At least a portion of the entrained debris may be deposited in the cleaner dust cup 108 for later disposal. The cleaner dust cup 108 can be configured to have a first emptying configuration and a second emptying configuration, wherein the cleaner dust cup 108 can be in the first emptying configuration when the vacuum cleaner 100 is undocked from the docking station 102 and can be in the second emptying configuration when the vacuum cleaner 100 is docked with the docking station 102. As such, the first emptying configuration may generally be referred to as a manual emptying configuration and the second emptying configuration may be generally referred to as an automated emptying configuration.

A user interface 112 can be disposed on and/or proximate to the handle 104 (e.g., within 10%, 15%, 20%, 25%, 35% or 50% of a maximum dimension of the handle 104). The user interface 112 may include one or more of a start toggle (e.g., for starting the suction motor 106), a cleaning behavior toggle (e.g., for increasing a suction power of the suction motor 106), a dust cup empty toggle (e.g., to transition the cleaner dust cup 108 to the manual emptying configuration), and/or any other toggle.

The docking station 102 includes a base 114, an up-duct 116 extending from the base 114, and receptacle 118 coupled to the up-duct 116. The receptacle 118 is configured to receive at least a portion of the vacuum cleaner 100. The base 114 includes a station dust cup 120 and a station suction motor 122. In some instances, the base 114 may also include a post motor filter 115, wherein exhaust from the station suction motor 122 is configured to pass through the post motor filter 115. The post motor filter 115 may be a high efficiency particulate air (“HEPA”) filter (e.g., a pleated HEPA filter).

The up-duct 116 includes an air channel 124 that is fluidly coupled to the station dust cup 120 and the station suction motor 122 such that the station suction motor 122, when activated, causes air to be drawn through the air channel 124 and into the station dust cup 120. The receptacle 118 includes a station inlet 126 that is fluidly coupled to the air channel 124 such that, when activated, the station suction motor 122 causes air to be drawn through the station inlet 126 and into the air channel 124. In other words, the up-duct 116 fluidly couples the station inlet 126 to the station suction motor 122 and the station dust cup 120.

As shown, the cleaner dust cup 108 includes a dust cup outlet 128 configured to fluidly couple to the station inlet 126 when the vacuum cleaner 100 is docked with the docking station 102 (e.g., when at least a portion of the vacuum cleaner 100 is received within the receptacle 118). When the station suction motor 122 is activated, air is caused to be drawn through the dust cup outlet 128 and into the station inlet 126. The dust cup outlet 128 may be configured to be selectively opened and closed when the vacuum cleaner 100 is docked to the docking station 102. When the dust cup outlet 128 is in the open configuration, the cleaner dust cup 108 is in the automated emptying configuration.

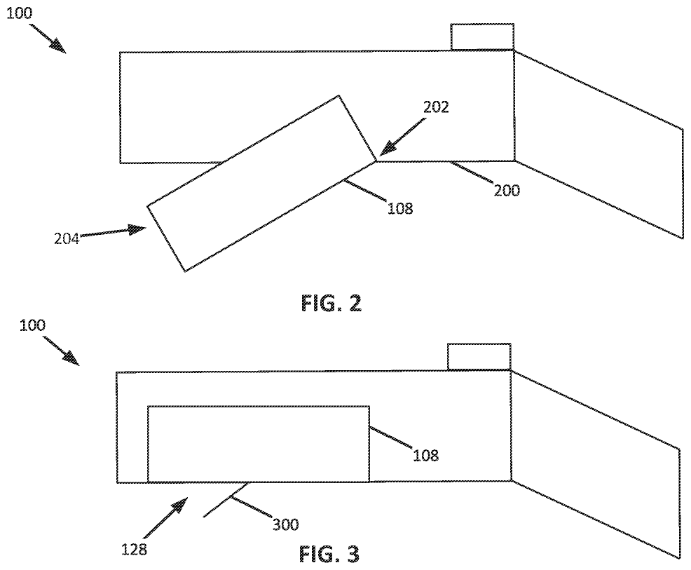

FIG. 2 shows a schematic example of the vacuum cleaner 100 having the cleaner dust cup 108 in the manual emptying configuration. As shown, the cleaner dust cup 108 is coupled (e.g., moveably coupled, removably coupled, and/or pivotally coupled) to a body 200 of the vacuum cleaner 100 such that the cleaner dust cup 108 is able to transition between a stowed configuration and the manual emptying configuration. For example, and as shown, the cleaner dust cup 108 can be pivotally coupled to the body 200 of the vacuum cleaner 100 at a pivot point 202 such that the cleaner dust cup 108 pivots from the stowed configuration to the manual emptying configuration. When in the manual emptying configuration, debris within the cleaner dust cup 108 may be emptied from a dust cup open end 204 of the cleaner dust cup 108. The dust cup open end 204 may be opposite the pivot point 202 of the cleaner dust cup 108. Such a configuration may encourage debris to be emptied from the dust cup open end 204 as a result of the pivotal movement of the cleaner dust cup 108.

FIG. 3 shows a schematic example of the vacuum cleaner 100 having the cleaner dust cup 108 in the stowed configuration and the dust cup outlet 128 in an open configuration. As shown, a dust cup door 300 may be configured to selectively open and close the dust cup outlet 128, selectively transitioning the dust cup outlet 128 between the open and closed configurations. The dust cup door 300 can be pivotally coupled to the cleaner dust cup 108 such that the dust cup door 300 pivots to selectively open and close the dust cup outlet 128. For example, when the vacuum cleaner 100 is docked with the docking station 102, airflow generated by the station suction motor 122 may cause the dust cup door 300 to pivot, opening the dust cup outlet 128 and allowing debris within the cleaner dust cup 108 to become entrained within the airflow. As such, the dust cup 108 may be generally described as being in an automated emptying configuration when the dust cup outlet 128 is in the open configuration.

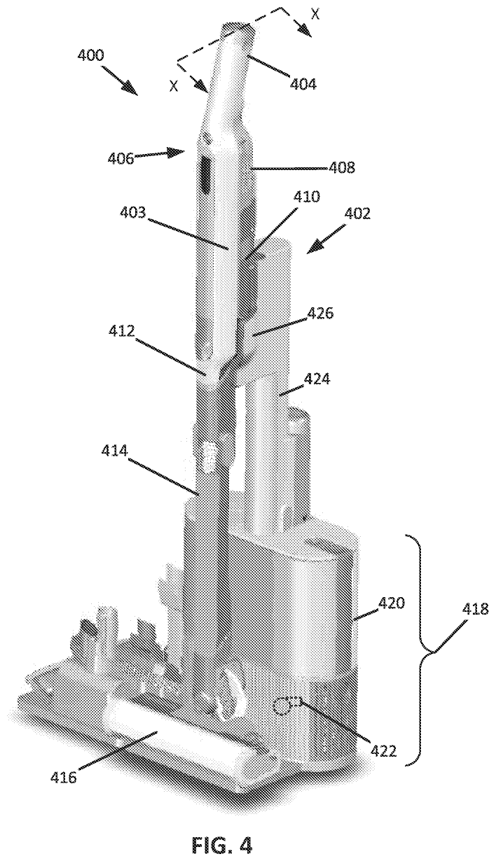

FIG. 4 shows a perspective view of a vacuum cleaner 400, which may be an example of the vacuum cleaner 100 of FIG. 1, and a docking station 402, which may be an example of the docking station 102 of FIG. 1.

The vacuum cleaner 400 includes a body 403, a handle 404, a cleaner user interface 406 proximate the handle 404, a cleaner suction motor 408, a cleaner dust cup 410 pivotally coupled to the body 403, and a cleaner inlet 412, the cleaner suction motor 408 being fluidly coupled to the cleaner dust cup 410 and the cleaner inlet 412. The cleaner inlet 412 may be configured to releasably couple to an accessory 414 (e.g., a cleaning wand). The accessory 414 may be configured to releasably couple to an additional accessory 416 (e.g., a floor nozzle).

The docking station 402 includes a base 418, a station dust cup 420 releasably coupled to the base 418, a station suction motor 422 disposed within the base 418, an up-duct 424 extending from the base 418, and a receptacle 426 coupled to the up-duct 424. The receptacle 426 is configured to receive at least a portion of the vacuum cleaner 400 such that the vacuum cleaner 400 releasably couples (docks) with the docking station 402. The receptacle 426 may also be configured to receive at least a portion of the accessory 414 such that the accessory 414 releasably couples (docks) with the docking station 402.

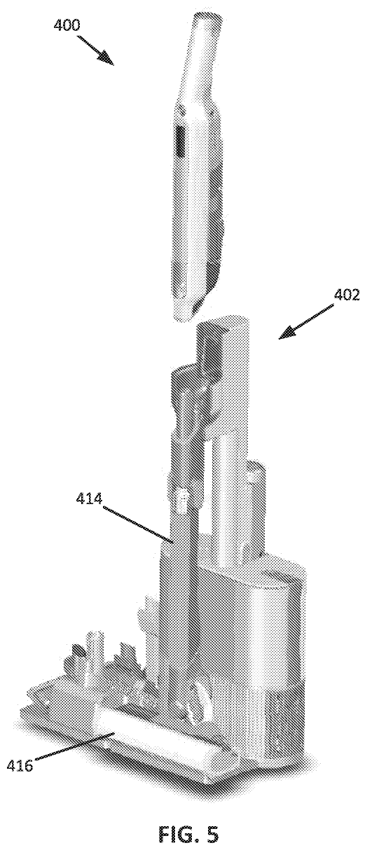

FIG. 5 shows a perspective view of the vacuum cleaner 400 and the docking station 402, wherein the vacuum cleaner 400 is undocked from the docking station 402. As shown, the vacuum cleaner 400 may be used independent of the accessories 414 and 416 and the accessories 414 and 416 may remain docked with the docking station 402 separate from the vacuum cleaner 400. When the vacuum cleaner 400 is undocked separately from the accessories 414 and 416, the accessories 414 and 416 may be undocked from the docking station 402 independent of the vacuum cleaner 400. In some instances, when the accessories 414 and 416 are not docked with the docking station 402, the vacuum cleaner 400 may be docked with the docking station 402 separately from the accessories 414 and 416.

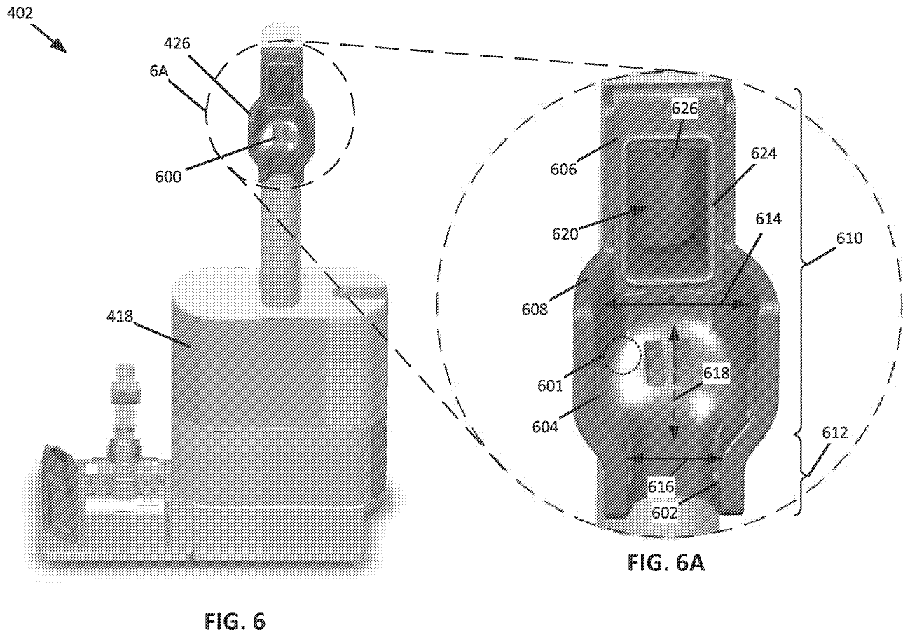

FIG. 6 shows a perspective view of the docking station 402 and FIG. 6A shows a magnified view corresponding to region 6A in FIG. 6. As shown, the receptacle 426 includes charging contacts 600 configured to electrically couple to the vacuum cleaner 400 (e.g., for charging one or more batteries of the vacuum cleaner 400), one or more accessory aligners 602, one or more cleaner aligners 604, and one or more dust cup aligners 606. In some instances, the docking station 402 may be configured to detect that the vacuum cleaner 400 is docked thereto using the charging contacts 600. Additionally, or alternatively, the receptacle 426 may include one or more sensors 601 (e.g., a tactile switch, a hall-effect sensor, and/or any other type of sensor) to detect that the vacuum cleaner 400 is docked thereto. In response to detecting the vacuum cleaner 400 is docked with the docking station 402, the docking station 402 may be caused to carry out an evacuation behavior. In some instances, the docking station 402 may carry out the evacuation behavior in response to detecting that the vacuum cleaner 400 is docked with the docking station 402 and in response to receiving a user input.

As shown, the receptacle 426 is defined by one or more receptacle sidewalls 608 that are shaped to follow a corresponding contour of the vacuum cleaner 400 and/or accessory 414 such that the receptacle 426 may be generally described as including a cleaner region 610 and an accessory region 612. For example, the receptacle 426 may have a first width 614 and a second width 616, wherein the first width 614 is greater than the second width 616. The second width 616 may be closer to the base 418 of the docking station 402 than the first width 614. In some instances, the second width 616 may generally correspond to a width of the accessory 414 (FIG. 4) and the first width 614 may correspond to a width of the vacuum cleaner 400 (FIG. 4). As such, the receptacle 426 may be generally described as being configured to receive at least a portion of the vacuum cleaner 400 and at least a portion of the accessory 414.

The one or more accessory aligners 602 are configured to engage (e.g., contact) the accessory 414 in order to align the accessory 414 relative to the receptacle 426. The one or more accessory aligners 602 may be grooves that are configured to receive a corresponding portion (e.g., an alignment protrusion) of the accessory 414. In some instances, at least a portion of the one or more accessory aligners 602 are configured to restrict movement of the of the accessory 414 to one or more predetermined axes when at least a portion of the accessory 414 is engaging the one or more accessory aligners 602. For example, at least a portion of the one or more accessory aligners 602 may be configured to restrict movement of the accessory 414 to an insertion/removal axis 618 of the receptacle 426 when at least a portion of the accessory 414 is engaging the one or more accessory aligners 602. The insertion/removal axis 618 may extend substantially (e.g., within 1°, 2°, 3°, 4°, or 5° of) parallel to a longitudinal axis of the up-duct 424.

The one or more cleaner aligners 604 are configured to engage (e.g., contact) the body 403 (FIG. 4) of the vacuum cleaner 400 in order to align the vacuum cleaner 400 relative to the receptacle 426. The one or more cleaner aligners 604 may be protrusions that are configured to be received within a corresponding groove in the vacuum cleaner 400 (e.g., in the body 403). In some instances, at least a portion of the one or more cleaner aligners 604 are configured to restrict movement of the vacuum cleaner 400 to one or more axes when at least a portion of the vacuum cleaner 400 engages the one or more cleaner aligners 604. For example, at least a portion of the one or more cleaner aligners 604 may be configured to restrict movement of the vacuum cleaner 400 to the insertion/removal axis 618 when at least a portion of the vacuum cleaner 400 is engaging the one or more cleaner aligners 604.

The one or more dust cup aligners 606 are configured to engage the cleaner dust cup 410 (FIG. 4) in order to align a dust cup outlet with a station inlet 620 of the receptacle 426. As shown, there may be a plurality of dust cup aligners 606 disposed on opposing sides of the station inlet 620. The one or more dust cup aligners 606 may be grooves configured to receive at least a portion of the cleaner dust cup 410. In some instances, at least a portion of the one or more dust cup aligners 606 are configured to restrict movement of the vacuum cleaner 400 to one or more axes when at least a portion of the cleaner dust cup 410 engages the one or more dust cup aligners 606. For example, at least a portion of the one or more dust cup aligners 606 may be configured to restrict movement of the vacuum cleaner 400 to the insertion/removal axis 618 when at least a portion of the cleaner dust cup 410 is engaging the one or more dust cup aligners 606. The dust cup aligners 606 may be further configured to urge the cleaner dust cup 410 into engagement with a seal 624 extending around a perimeter of the station inlet 620. The seal 624 can be resiliently deformable such that, when the vacuum cleaner 400 is received within the receptacle 426, the seal 624 is at least partially compressed. For example, the seal 624 may include thermoplastic polyurethane (“TPU”).

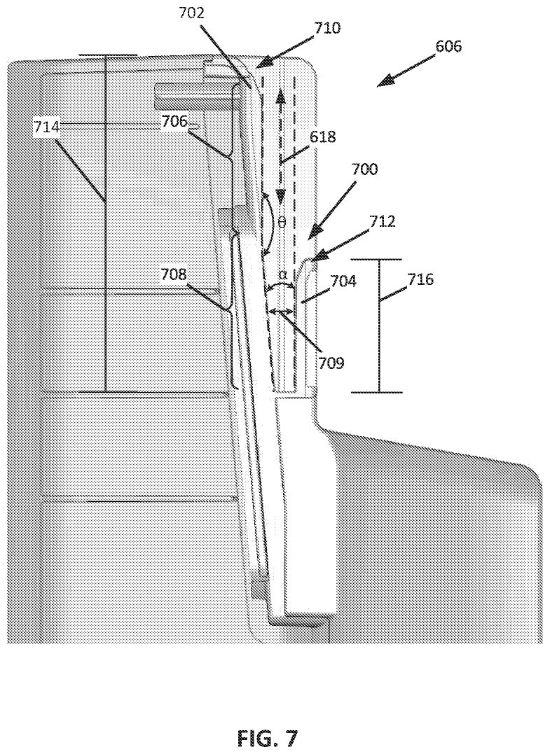

With reference to FIG. 7, which shows a cross-sectional view of a portion of the receptacle 426, the one or more dust cup aligners 606 may include a dust cup aligner groove 700 defined by a first groove sidewall 702 and a second groove sidewall 704. The first and second groove sidewalls 702 and 704 may be configured to encourage formation of a seal between the seal 624 (FIG. 6A) and the cleaner dust cup 410 and/or mitigate wear on the seal 624 resulting from repeated docking and undocking of the vacuum cleaner 400 with the docking station 402. The first groove sidewall 702 may include a first sidewall portion 706 and a second sidewall portion 708, the first sidewall portion 706 intersecting the second sidewall portion 708 to form a sidewall portion angle θ. The sidewall portion angle θ may be an obtuse angle that extends between surfaces of the first and second sidewall portions 706 and 708 that face the second groove sidewall 704. The second sidewall portion 708 may form a groove angle α with the second groove sidewall 704 such that a separation distance 709 extending between the second sidewall portion 708 and the second groove sidewall 704 decreases in a direction of the base 418 of the docking station 402. In other words, the dust cup aligner groove 700 may include a tapering region that tapers in a direction of the base 418.

The groove angle α extends from a surface of the second sidewall portion 708 that faces the second groove sidewall 704 to the second groove sidewall 704. The groove angle α may be, for example, in a range of 1° to 20°. By way of further example, the groove angle α may be, for example, in a range of 5° to 15°. By way of still further example, the groove angle α may be, for example, about (e.g., within 1%, 2%, 3%, 4,% or 5% of) 10°.

The first and/or second groove sidewall 702 and/or 704 may include a chamfered region 710 and/or 712 configured to encourage insertion of at least a portion of the cleaner dust cup 410 (FIG. 4) into the dust cup aligner groove 700. The first groove sidewall 702 has a first sidewall height 714 and the second groove sidewall 704 has a second sidewall height 716. The first sidewall height 714 may be greater than the second sidewall height 716. As such, movement of the vacuum cleaner 400 along the insertion/removal axis 618 may be restrained for only a portion of the dust cup aligner groove 700 (e.g., the portion of the dust cup aligner groove 700 extending between the first and second groove sidewalls 702 and 704).

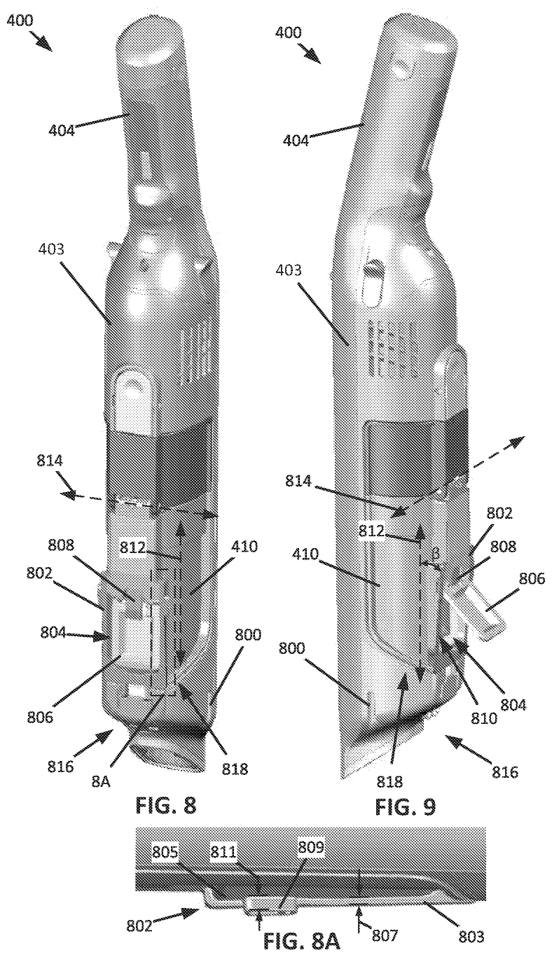

FIGS. 8 and 9 show perspective views of the vacuum cleaner 400. As shown, the body 403 of the vacuum cleaner 400 includes one or more cleaner alignment grooves 800 configured to cooperate with the docking station 402 (e.g., the one or more cleaner aligners 604 (FIG. 6A) of the receptacle 426) and the cleaner dust cup 410 includes a dust cup alignment protrusion 802 configured to cooperate with the docking station 402 (e.g., the dust cup aligners 606 (FIG. 6A)). The dust cup alignment protrusion 802 may include a dust cup outlet 804 that is configured to be selectively opened and closed by a dust cup door 806 such that debris within the cleaner dust cup 410 may selectively pass therethrough.

As shown, the dust cup door 806 is configured to transition between a closed position (FIG. 8) and an open position (FIG. 9). For example, the dust cup door 806 can be pivotally coupled to the cleaner dust cup 410 (e.g., the dust cup alignment protrusion 802) such that the dust cup door 806 pivots between the open and closed positions. The dust cup door 806 may be biased (e.g., using a spring such as a torsion spring) towards the closed position. When the dust cup door 806 is in the open position, the cleaner dust cup 410 may generally be described as being in an automated emptying configuration.

The vacuum cleaner 400 (e.g., the cleaner dust cup 410) may include a retainer 808. The retainer 808 may be moveably (e.g., slidably) coupled to the dust cup alignment protrusion 802, wherein the retainer 808 is configured to transition between a locked position (FIG. 8) and an unlocked position (FIG. 9). When the retainer 808 is in the locked position, the dust cup door 806 is prevented from moving from the closed position to the open position (e.g., pivotal movement of the dust cup door 806 may be substantially prevented). When the retainer 808 is in the unlocked position, the dust cup door 806 is capable of moving from the closed position to the open position. The retainer 808 may be biased (e.g., using a spring such as a compression spring) towards the locked position.

The retainer 808 may be transitioned from the locked position to the unlocked position when the vacuum cleaner 400 is being docked with the docking station 402. For example, the receptacle 426 may include an actuation protrusion 626 (FIG. 6A) that extends transverse to (e.g., perpendicular to) the insertion/removal axis 618. The actuation protrusion 626 is configured to engage (e.g., contact) the retainer 808 when the vacuum cleaner 400 is being received by the receptacle 426. Engagement of the actuation protrusion 626 with the retainer 808 causes the retainer to transition (e.g., slide) from the locked position to the unlocked position when the vacuum cleaner 400 is docked with the docking station 402.

The dust cup alignment protrusion 802 is configured to cooperate with the dust cup aligners 606. For example, the dust cup alignment protrusion 802 may have a shape (e.g., a wedged shape) that generally corresponds to the shape of the dust cup aligner groove 700 (FIG. 7). For example, the shape of the dust cup alignment protrusion 802 may be such that second groove sidewall 704 engages (e.g., contacts) the dust cup alignment protrusion 802, urging the dust cup alignment protrusion 802 into engagement (e.g., contact) with the seal 624 (FIG. 6A). Engagement between the seal 624 and the dust cup alignment protrusion 802 may at least partially compress the seal 624. For example, a seal engaging surface 810 of the dust cup alignment protrusion 802 may come into engagement with the seal 624 forming an at least partial seal. Formation of a partial seal may mitigate debris pluming when the cleaner dust cup 410 is being emptied.

In some instances, and with additional reference to FIG. 8A (which is magnified view generally corresponding to region 8A in FIG. 8), the dust cup alignment protrusion 802 may further include an alignment lip 803 that extends outwardly from a protrusion sidewall 805 of the dust cup alignment protrusion 802 by a first extension distance 807. The dust cup alignment protrusion 802 may include a plurality of alignment lips 803, wherein each alignment lip 803 extends along opposing longitudinal sides of the dust cup alignment protrusion 802. The alignment lip 803 may be configured to engage at least a portion of the dust cup aligners 606. In some instances, the alignment lip 803 may include at least a portion of the seal engaging surface 810 of the dust cup alignment protrusion 802. The dust cup alignment protrusion 802 may include (in addition to or in the alternative to the alignment lip 803) an alignment projection 809. The alignment projection 809 may extend from the protrusion sidewall 805 by a second extension distance 811, the second extension distance 811 being greater than the first extension distance 807. The alignment projection 809 may be configured to engage at least a portion of the dust cup aligners 606. In some instances, the alignment projection 809 may include at least a portion of the seal engaging surface 810 of the dust cup alignment protrusion 802.

As shown, the seal engaging surface 810 of the dust cup alignment protrusion 802 forms a protrusion angle β with a cleaner longitudinal axis 812. The protrusion angle β may generally correspond to the groove angle α (FIG. 7). The protrusion angle β may be, for example, in a range of 1° to 20°. By way of further example, the protrusion angle β may be, for example, in a range of 5° to 15°. By way of still further example, the protrusion angle β may be, for example, about (e.g., within 1%, 2%, 3%, 4,% or 5% of) 10°.

The cleaner dust cup 410 is pivotally coupled to the body 403 of the vacuum cleaner 400 about a dust cup pivot axis 814. The cleaner dust cup 410 is configured to pivot about the dust cup pivot axis 814 from a stowed configuration to a manual emptying configuration. As shown, when in the stowed configuration, the cleaner dust cup 410 extends along the cleaner longitudinal axis 812 between an inlet end 816 of the body 403 and the handle 404. When the cleaner dust cup 410 pivots to the manual emptying position, an open end 818 of the cleaner dust cup 410 is exposed. As shown, the open end 818 is received within the body 403 when the cleaner dust cup 410 is in the stowed configuration. As such, the cleaner dust cup 410 may generally be described as being configured to pivot such that the open end 818 is selectively received within the body 403. The open end 818 and the dust cup outlet 804 can be on different sides of the cleaner dust cup 410.

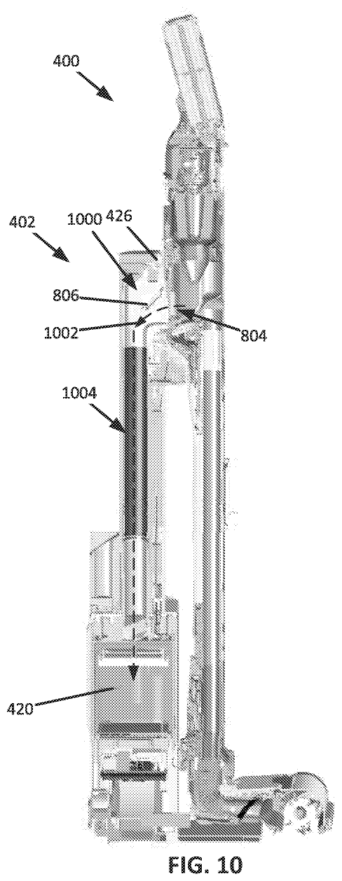

FIG. 10 shows a cross-sectional view of the vacuum cleaner 400 docked with the docking station 402 of FIG. 4 taken along the line X-X of FIG. 4. As shown, the dust cup door 806 is in the open position. The dust cup door 806 can be transitioned from the closed position to the open position in response to the station suction motor 422 (FIG. 4) being activated. For example, the airflow generated by the station suction motor 422 may urge the dust cup door 806 towards the open position. When the station suction motor 422 is deactivated, the dust cup door 806 may transition to the closed position (e.g., as a result of gravity and/or a biasing force). When the dust cup door 806 is in the open position, at least a portion of the dust cup door 806 passes through the station inlet 620 and is at least partially received within a receptacle cavity 1000 of the receptacle 426. In other words, when the dust cup outlet 804 is open, at least a portion of the dust cup door 806 is received within the receptacle cavity 1000.

The airflow generated by the station suction motor 422 may flow along an evacuation flow path 1002. As shown, the evacuation flow path 1002 extends from the cleaner dust cup 410 into the receptacle cavity 1000 through an air channel 1004 of the up-duct 424 and into the station dust cup 420.

An example of a vacuum cleaner, consistent with the present disclosure, may include a body and a dust cup coupled to the body. The dust cup may include an open end that is configured to be selectively received within the body and a dust cup outlet that is configured to be selectively opened and closed.

In some instances, the dust cup may further include a dust cup door configured to selectively open and close the dust cup outlet. In some instances, the dust cup door may be pivotally coupled to the dust cup. In some instances, the dust cup may further include a retainer configured to transition between a locked position and an unlocked position, wherein pivotal movement of the dust cup door is substantially prevented when the retainer is in the locked position. In some instances, the retainer may be biased towards the locked position. In some instances, the dust cup may further include a dust cup alignment protrusion configured to cooperate with a docking station, the dust cup alignment protrusion including the dust cup outlet. In some instances, the body may include an alignment groove configured to cooperate with a docking station. In some instances, the dust cup outlet and the open end may be on different sides of the dust cup.

An example of a cleaning system, consistent with the present disclosure, may include a vacuum cleaner having a body and a cleaner dust cup coupled to the body and a docking station, the vacuum cleaner configured to dock with the docking station. The cleaner dust cup may include an open end that is configured to be selectively received within the body and a dust cup outlet that is configured to be selectively opened and closed, the dust cup outlet and the open end being on different sides of the cleaner dust cup. The docking station may include a base having a suction motor and a station dust cup, an up-duct extending from the base, and a receptacle having a station inlet, the receptacle being configured to receive at least a portion of the vacuum cleaner, the up-duct fluidly couples the station inlet to the suction motor and the station dust cup.

In some instances, the station inlet may be configured to fluidly couple with the dust cup outlet when the vacuum cleaner is docked with the docking station. In some instances, the cleaner dust cup may further include a dust cup door configured to selectively open and close the dust cup outlet. In some instances, the receptacle may include a receptacle cavity, the receptacle cavity being configured to receive at least a portion of the dust cup door when the dust cup outlet is open. In some instances, the dust cup door may be configured to pivot to selectively open and close the dust cup outlet and an airflow generated by the suction motor pivots the dust cup door to open the dust cup outlet. In some instances, the cleaner dust cup may further include a retainer configured to transition between a locked position and an unlocked position, wherein movement of the dust cup door is substantially prevented when the retainer is in the locked position. In some instances, the receptacle may include an actuation protrusion configured to transition the retainer from the locked position to the unlocked position when the vacuum cleaner is docked with the docking station. In some instances, the actuation protrusion may extend transverse to an insertion/removal axis of the receptacle. In some instances, the retainer may be biased towards the locked position. In some instances, the receptacle may include a dust cup aligner configured to align the dust cup outlet with the station inlet. In some instances, the dust cup aligner may include a groove, the groove including a tapering region that tapers in a direction of the base. In some instances, the receptacle may include a cleaner aligner. In some instances, the vacuum cleaner may include an alignment groove configured to cooperate with the cleaner aligner. In some instances, the dust cup may be pivotally coupled to the body.

In some instances, a docking station for a vacuum cleaner may include one or more odor control assemblies to control the odor in a dust cup in the docking station. The odor control assemblies may include an adjustment member that can be transitioned to vary an amount of fragrance particles output by the odor control assembly during use, e.g., during evacuation of the dust cup in the vacuum cleaner into the dust cup in the docking station.

In more detail, the fragrance particles may be provided by a fragrance member that is coupled to the adjustment member, with the fragrance member providing at least one fragrance air path. The adjustment member can adjust the cross-sectional size of the opening to the fragrance air path by rotating the odor control assembly to cover or expose the air path, thereby regulating the amount of air that may be drawn through the fragrance member. The air traveling through the fragrance air path may then cause fragrance particles to become airborne. The odor control assembly may then output the fragrance particles into the dust cup in the docking station. The air communicated through the air path of the fragrance member can be provided from a motor, for example, a suction motor disposed in the docking station for evacuating debris from the dust cup in the vacuum cleaner into the dust cup in the docking station. The temperature of the air communicated across the motor, and/or the velocity of the air communicated across the motor, may be advantageously utilized to ensure that a predetermined amount of fragrance particles get output by the odor control assembly.

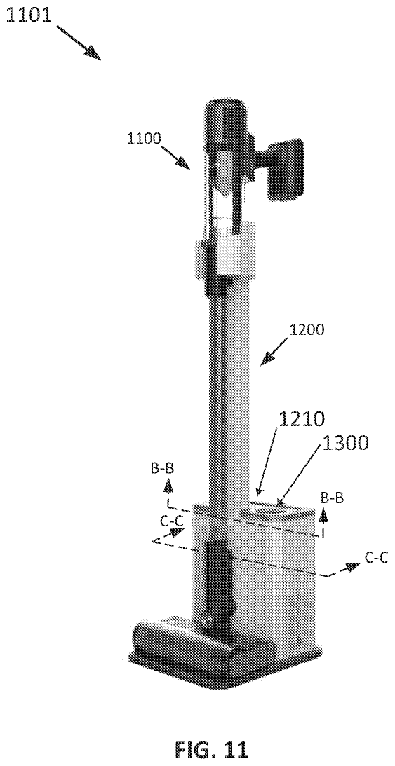

FIG. 11 is a perspective view of a cleaning system 1101 having a vacuum cleaner 1100, which may be an example of the vacuum cleaner 100 of FIG. 1, removably coupled (docked) to a docking station 1200, which may be an example of the docking station 102 of FIG. 1. The cleaning system 1101 of FIG. 11 may further include station dust cup 1210, and station dust cup 1210 may further include an odor control assembly 1300.

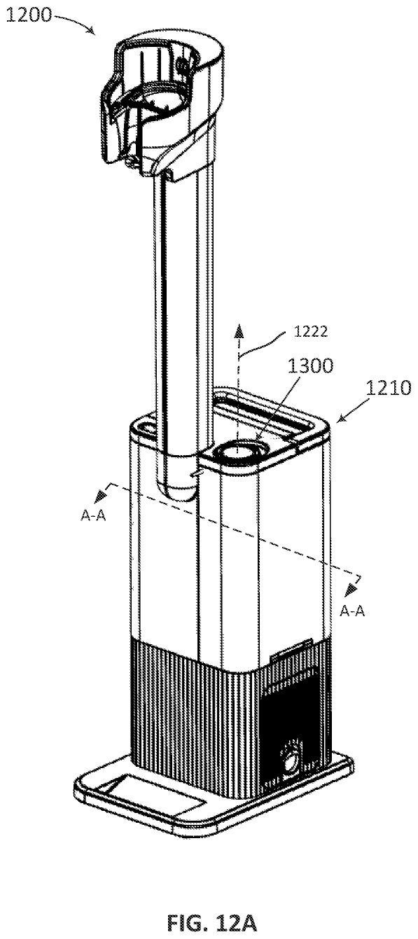

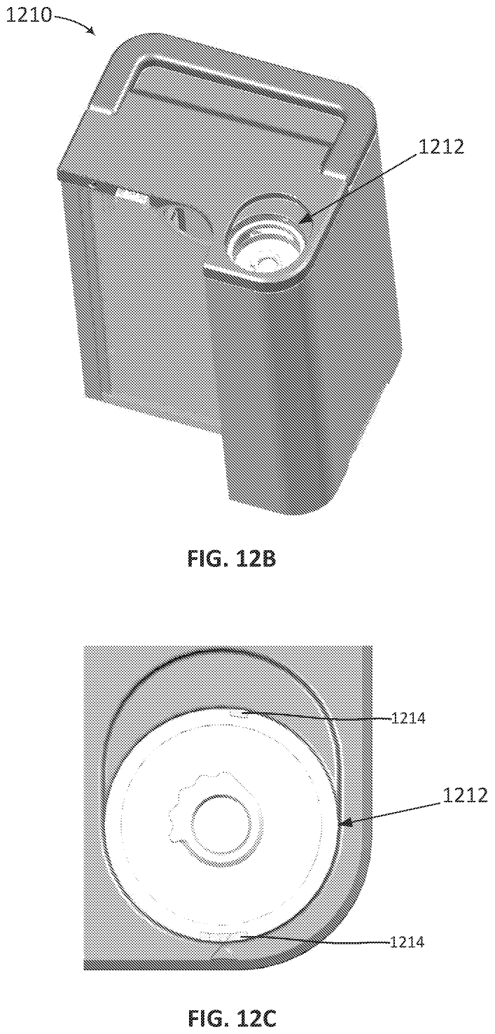

FIG. 12A is a perspective view of the docking station 1200, with the vacuum cleaner 1100 undocked from the docking station. The odor control assembly 1300 may be removably coupled to the station dust cup 1210. For example, the odor control assembly 1300 may be at least partially disposed in the station dust cup 1210, and more particularly, in the odor control cavity 1212 (FIG. 12B) in a top surface of the station dust cup 1210. A closeup view of the odor control cavity in the top surface of the station dust cup 1210 is shown in FIG. 12C. FIG. 12C also shows protrusions 1214 that retain the odor control assembly 1300 in the station dust cup 1210.

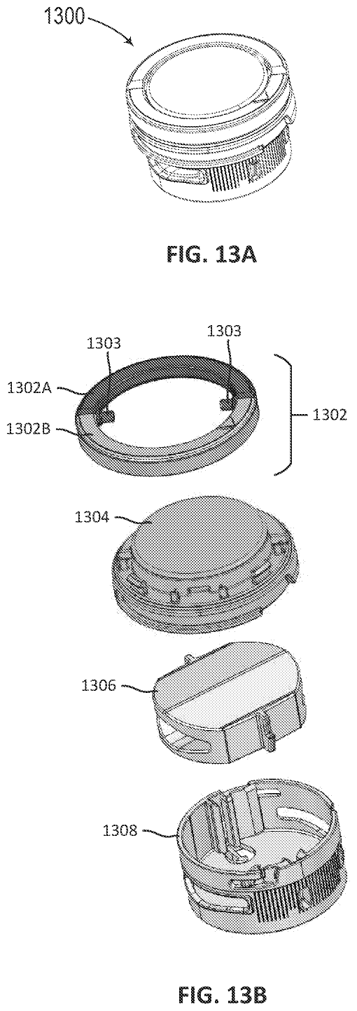

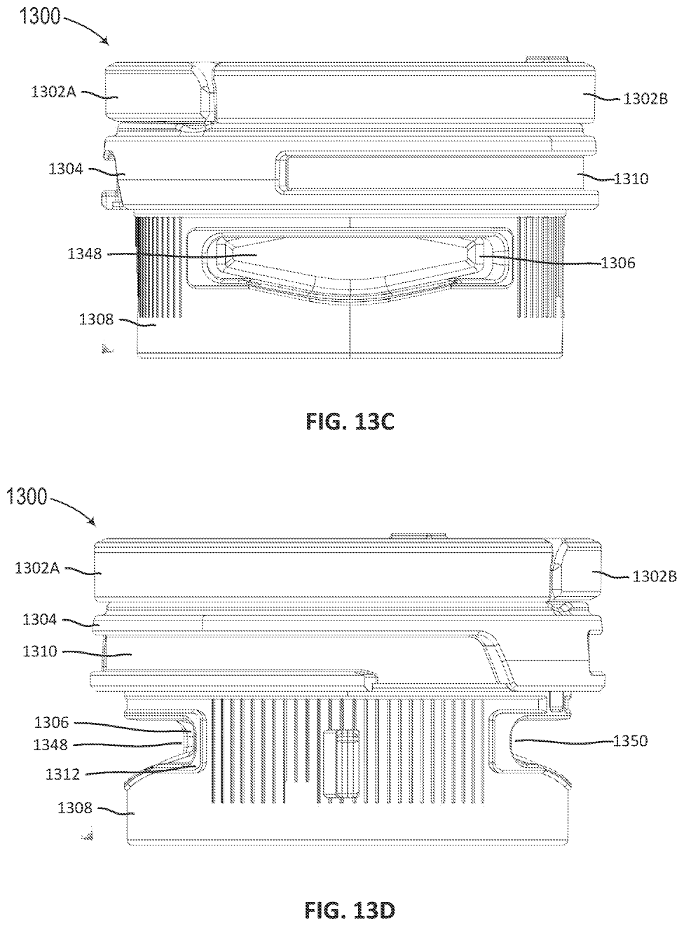

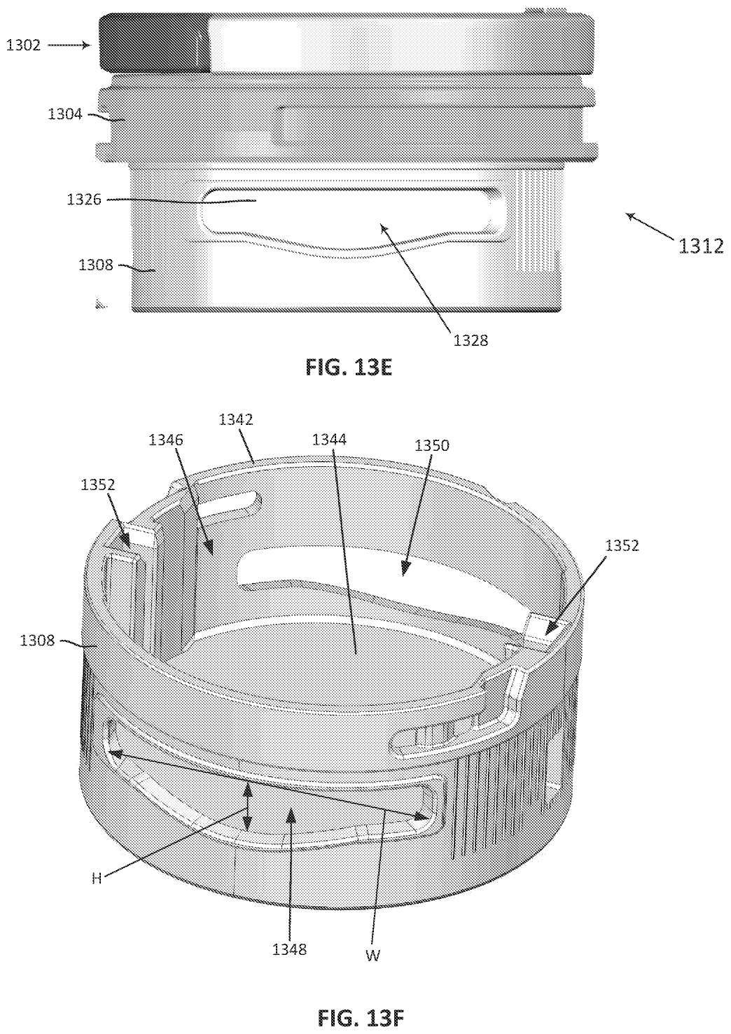

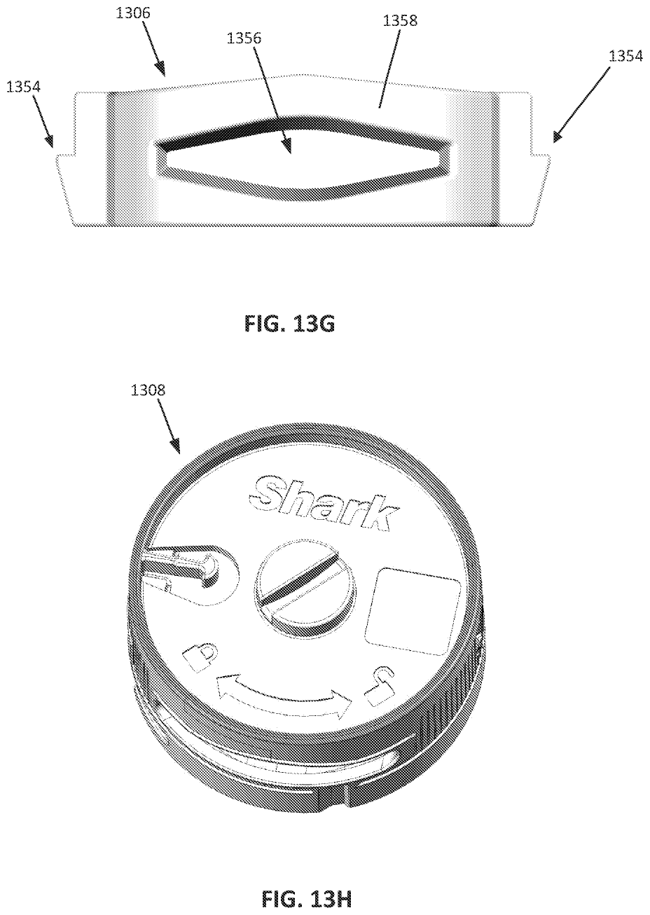

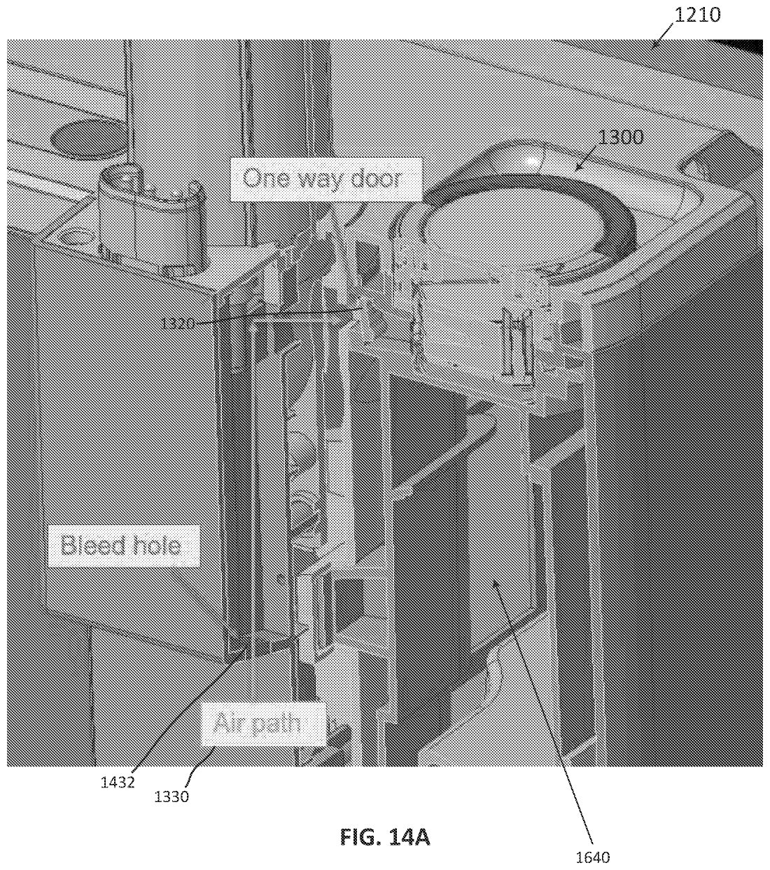

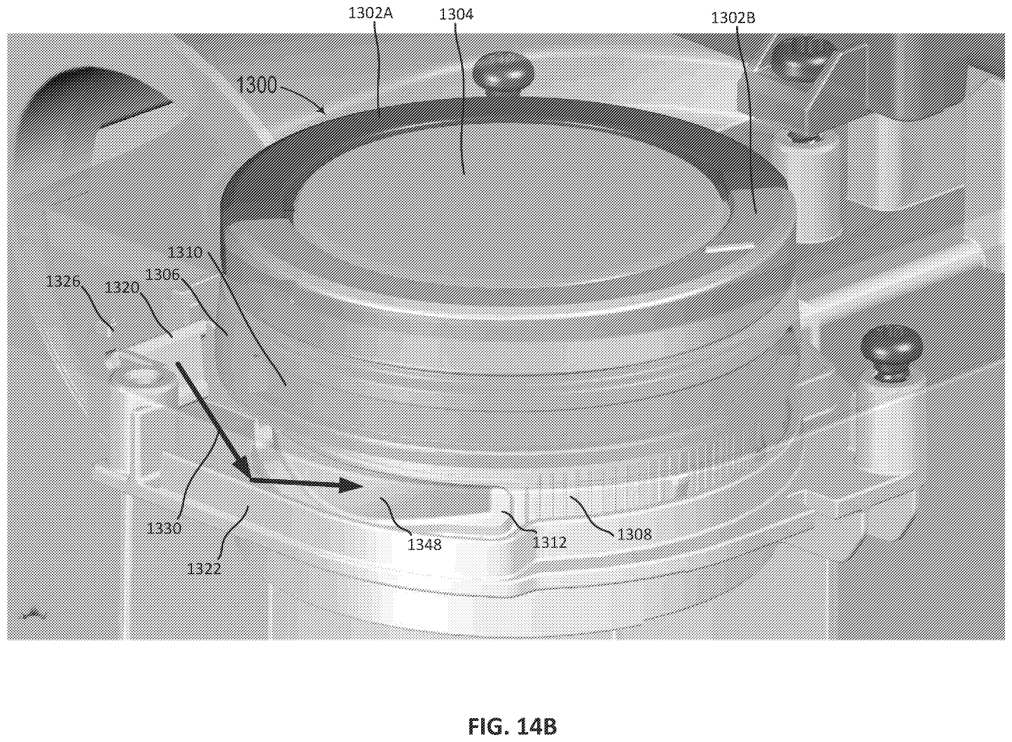

With reference to FIG. 13E, one example of an odor control assembly 1300 consistent with the present disclosure is generally illustrated. The odor control assembly 1300 may include a dial body 1312 configured to be removably secured to the station dust cup 1210 and configured to receive one or more scent pucks 1306. The dial body 1312 may have a generally circular cross-section and may be configured to generally form one or more seals with the station dust cup 1210 and may optionally define a fragrance cavity 1326 configured to receive and generally enclose the scent puck 1306. The dial body 1312 and/or the puck cartridges 1308 may define one or more fragrance passageways 1328 configured to allow air to flow over/past the puck cartridges 1308 to transfer fragrance particles into the air to form the fragranced air.

Turning now to FIG. 13B, an exploded view of one example of the odor control assembly 1300 is generally illustrated. The dial body 1312 may include a puck cartridge 1308 and a puck cap 1304. The puck cartridge 1308 and puck cap 1304 may be configured to be removable coupled to each other to at least partially form the fragrance cavity 1326 and the fragrance passageway 1328. In the illustrated example, the puck cartridge 1308 includes an entrance 1348 and an exit 1350 (see FIG. 13F) to the fragrance passageway 1328. Atmospheric air may flow through the entrance 1348, across the scent puck 1306, and out of the exit. The puck cap 1304 may optionally include one or more rotatable sections 1302A that functions as a handle or D-ring to aid in insertion and removal of the odor control assembly 1300. The rotatable section 1302A may be coupled to puck cap 1304, for example, by way of one or more hinges 1303 or the like. The puck cap 1304 may also optionally include a fixed ring 1302B secured to the puck cap 1304.

As noted above, the puck cartridge 1308 and puck cap 1304 may be configured to be removable coupled to each other to at least partially form the fragrance cavity 1326 and the fragrance passageway 1328. The puck cartridge 1308 and puck cap 1304 may be removably secured to each other in any manner known to those skilled in the art such as, but not limited to, threaded connections, tabs, detents, clips, or the like.

One benefit of the removable connection between the puck cartridge 1308 and puck cap 1304 is that is allows for the replacement of the puck cartridge 1308 and the scent puck 1306 to be accomplished without the user having to touch the scent puck 1306 and without having to replace the entire odor control assembly 1300. In particular, when the user desires to replace the scent puck 1306, the user may purchase the puck cartridge 1308 which is preloaded with the scent puck 1306. The user may then disconnect the puck cartridge 1308 (which includes the scent puck 1306) from the puck cartridge 1308 and then connect a new puck cartridge 1308 (in which the scent puck 1306 is preloaded therein) to the existing puck cartridge 1308.

Turning now to FIG. 13F, one example of the puck cartridge 1308 is generally illustrated. The puck cartridge 1308 may include one or more sidewalls 1342, for example, extending upwardly from a base 1344. The sidewall 1342 (and optionally the base 1344) may define a puck chamber 1346 configured to receive the scent puck 1306. The puck chamber 1346 may be the same as the fragrance cavity 1326 or may define a portion of the fragrance cavity 1326. The sidewall 1342 may also at least partially define the entrance 1348 and exit 1350 to the fragrance passageway 1328. In the illustrated example, the entrance 1348 and exit 1350 to the fragrance passageway 1328 are generally aligned 180 degrees opposite each other; however, it should be appreciated that the entrance 1348 and exit 1350 may be aligned at any other angle.

The sidewall 1342 may optionally include one or more puck alignment features 1352. The puck alignment features 1352 are configured to align the scent puck 1306 relative to the entrance 1348 and exit 1350. In the illustrated example, the puck alignment features 1352 include grooves configured to receive corresponding tabs 1354 (FIG. 12) of the scent puck 1306 and to align the passageway 1356 extending through the body 1358 of the scent puck 1306 with the entrance 1348 and exit 1350. The entrance 1348, exit 1350, and the passageway 1356 may collectively define (at least in part) the fragrance passageway 1328. The height H of the entrance 1348 and/or exit 1350 may vary across the width W. In particular, the height H may be less proximate one or more of the ends of the width and larger in-between (e.g., the middle). The varying height H may facilitate the adjustment of the airflow through the fragrance passageway 1328 as the odor control assembly 1300 is rotated. The passageway 1356 extending through the body 1358 of the scent puck 1306 may include a through hole aligned with the entrance and the exit of the fragrance air path. The through hole may define a passage through the scent puck 1306 which is surrounded by the scent puck 1306 and having an entrance and an outlet. The through hole may also have a cross-section that corresponds to the cross-section of the entrance 1348, exit 1350. A benefit of the through hole in the scent puck 1306 is that it increases the surface area available to transfer fragrance particles into the air flowing through the scent puck 1306.

With reference to FIG. 13H, The bottom surface of the puck cartridge 1308 may optionally include a slot or the like that allows for easy disconnection of the puck cartridge 1308 from the puck cap 1304. This may allow a user to remove the puck cartridge 1308 without having to touch the puck cartridge 1308.

In some instances, the odor control assembly 1300 may be implemented as shown in FIG. 13A and FIG. 13B. The odor control assembly 1300 includes an adjustment assembly 1302, composed of pivoting adjustment member 1302A and fixed ring 1302B, a puck cap 1304, a scent puck 1306, and a puck cartridge 1308. The adjustment assembly 1302, puck cap 1304, scent puck 1306, and puck cartridge 1308 are removably coupled to each other to form odor control assembly 1300. The adjustment assembly 1302, puck cap 1304, scent puck 1306, and puck cartridge 1308 may be removably secured to each other in any manner known to those skilled in the art such as, but not limited to, threaded connections, tabs, detents, clips, or the like.

The adjustment assembly 1302 may be configured to allow for a user to adjust an amount of fragrance particles introduced into a dirty air passageway of the station dust cup 1210 based on rotational movement of the adjustment assembly 1302 about a rotational axis 1222 (see FIG. 12A). The adjustment assembly 1302 may be configured to transition the adjustment assembly 1302 between a plurality of user-selected positions.

When the user applies a rotational force to the adjustment assembly 1302, the odor control assembly 1300 is caused to rotate as a unit relative to the cleaning system 1101 and/or station dust cup 1210, thereby controlling the amount of input air allowed to pass through the odor control assembly 1300 into the station dust cup 1210. The user-selectable positions can include at least a fully open position to release a first predetermined amount of fragrance from the fragrance member into the station dust cup 1210, and a closed position to substantially prevent and/or minimize the amount of fragrance being released into the station dust cup 1210 of the cleaning system 1101. The user may rotate the adjustment assembly 1302, and thereby the odor control assembly 1300, to any position between the fully open position and the substantially closed position to achieve the desired amount of fragrance released into the station dust cup 1210.

The plurality of user-selectable positions may include a release position. The release position may be at a location that is rotationally outside of the user-selectable positions that are used for adjustment of the fragrance particle output. In the release position, the adjustment assembly 1302 may be configured to decouple from the station dust cup 1210 based on a pulling force supplied by a user along an axis that extends substantially parallel (e.g., coaxially) with the rotational axis 1222. The adjustment assembly 1302 and the scent puck 1306 can be decoupled from the station dust cup 1210 in the release position. The adjustment assembly 1302 and scent puck 1306 may be secured together such that the adjustment assembly 1302 and the scent puck 1306 remain coupled together when the adjustment assembly 1302 is decoupled from the station dust cup 1210. In some instances, the release position may allow for the entire odor control assembly 1300 to decouple from the station dust cup as a unit.

The adjustment assembly 1302 may include pivoting adjustment member 1302A that can pivot about pins 1303, which are inserted into corresponding holes (not shown) in puck cap 1304. The user may rotate the pivoting adjustment member 1302A away from the puck cap 1304 to facilitate rotating the adjustment assembly 1302. In addition, the user may rotate the pivoting adjustment member 1302A to allow for removal and replacement of the scent puck 1306.

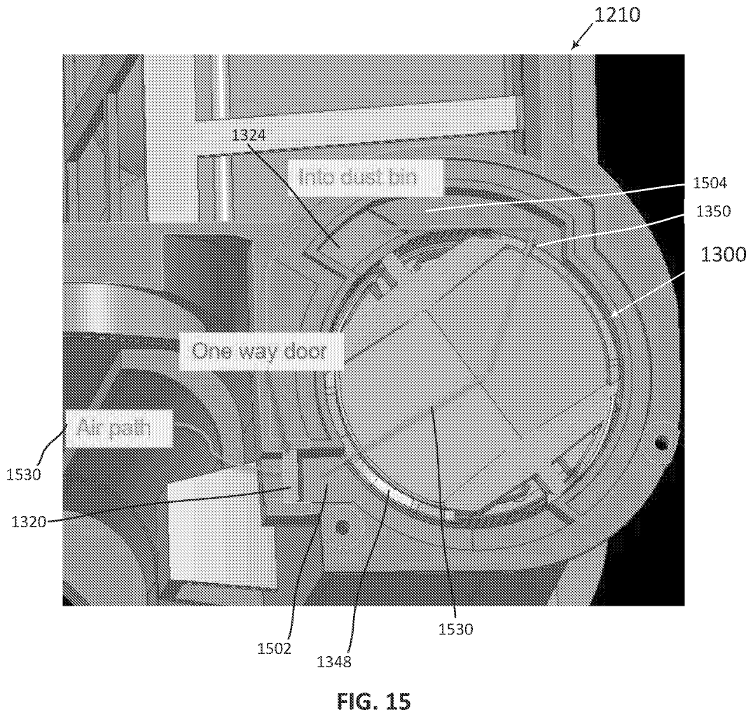

FIG. 13C is a front view of the odor control assembly 1300, and FIG. 13D shows a side view of the odor control assembly 1300. FIGS. 13C and 13D illustrate how the adjustment assembly 1302 controls the amount of fragrance particles allowed to pass through the odor control assembly 1300 into the station dust cup 1210, and thereby the amount of fragrance particles released into the station dust cup 1210. As can be seen in FIGS. 13C and 13D, puck cartridge 1308 and scent puck 1306 having opening 1314 on the left side of puck cartridge 1308 and scent puck 1306 and opening 1316 on the right side of puck cartridge 1308 and scent puck 1306. In the fully open position, openings 1314 and 1316 align with an inlet port (1502 in FIG. 15) and an output port (1504 in FIG. 15), respectively. As the adjustment assembly 1302 is rotated towards the minimum position, the openings 1314 and/or 1316 rotate away from the inlet port 1502 and/or the outlet port 1504, until when fully rotated, the inlet port 1502 and/or outlet port 1504 are substantially blocked (i.e., openings 1314 and/or 1316 are not aligned with the inlet port 1502 and/or outlet port 1504). The minimum and maximum rotation positions of the adjustment assembly 1302 may be restricted by the interaction of cam 1310 and matching protrusions 1214 in odor control cavity 1212. Of course, it should be appreciated that the amount of fragrance particles dispensed from the odor control assembly 1300 into the station dust cup 1210 may be adjusted in any manner known to those skilled in the art in view of the instant application.

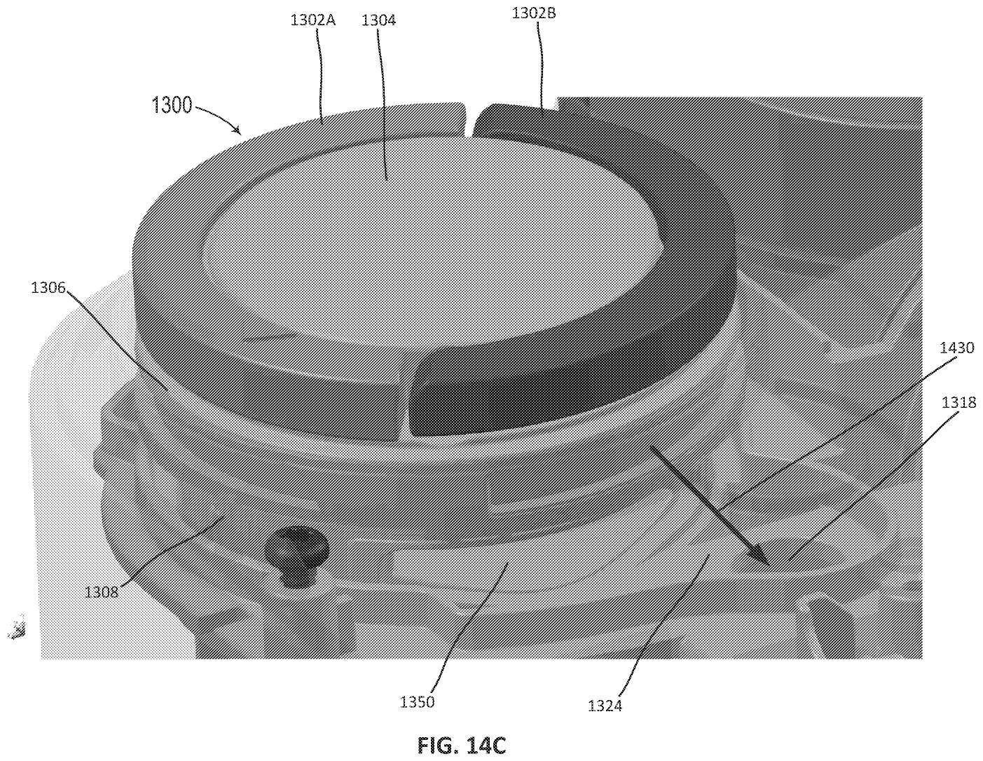

FIG. 14A is a front cross-sectional view of an odor control assembly 1300 mounted in the station dust cup 1210 within the docking station 1200 taken along line A-A of FIG. 12A, showing the inlet air path 1330 for clean air from external to the docking station 1200 to the odor control assembly 1300. In some embodiments, the docking station 1200 may include a bleed hole 1432 that is fluidly coupled to the inlet air path 1330 such that the station suction motor, e.g., station suction motor 122 from FIG. 1, when activated, causes air to be drawn through the inlet air path 1330 and into the station dust cup 1210 through the odor control assembly 1300. The inlet air path 1330 may also include a backflow preventor 1320 which is configured to generally seal off the odor control assembly 1300 from atmospheric air (e.g., when the station suction motor 122 is turned off). In one example, the backflow preventor 1320 may include one or more one-way valves or doors, which may be further configured to return to a seated/sealed position when the flow rate and/or pressure of the air through the inlet air path 1330 falls below a threshold (e.g., the station suction motor 122 is turned off) to minimize or otherwise substantially prevent air from escaping from the inlet air path 1330 (e.g., substantially prevent air flow from the odor control assembly 1300 to the atmosphere). The backflow preventor 1320 may increase the lifespan of the odor control assembly 1300 by minimizing exposure of the odor control assembly 1300 to atmospheric air, thereby minimizing the amount of fragrance particles dispensed by the odor control assembly 1300 when the flow rate and/or pressure within the inlet air path 1330 is below a threshold.

In some other embodiments, the bleed hole and air path 1330 may be eliminated. For example, the odor control assembly 1300 (e.g., the puck cartridge 1308 and/or scent puck 1306) may be at least partially exposed and/or disposed within the debris cavity 1640. Air may be drawn through the odor control assembly 1300 (e.g., the puck cartridge 1308 and/or scent puck 1306), for example, by suction from the station suction motor 122 and/or the cleaner suction motor 106. The odor control assembly 1300 may also dispense fragrance particles into the debris cavity 1640 without the use suction from the station suction motor 122 and/or the cleaner suction motor 106. For example, the odor control assembly 1300 may diffuse fragrance particles into the debris cavity 1640 by virtue of the odor control assembly 1300 being at least partially disposed within the debris cavity 1640.

FIG. 14B is a front perspective view of an odor control assembly 1300 mounted in a station dust cup 1210 that shows the inlet air path 1330 between the one-way door 1320 and the entrance 1348 of the odor control assembly 1300 (e.g., puck cartridge 1308). The inlet air path 1330 is defined, at least in part, by a chamber formed by sidewall 1322 that at least partially surrounds the odor control assembly 1300 (e.g., puck cartridge 1308) and is fluidly coupled to the station suction motor. The cross-sectional size of the entrance 1348 that is in fluid communication with the inlet air path 1330 will increase or decrease depending on the rotational position of the odor control assembly 1300 relative to the docking station 1200 and/or station dust cup 1210, as selected by the user. The example of FIG. 14B shows the odor control assembly 1300 in an open position. As the odor control assembly 1300 is rotated, the cross-sectional size of the entrance 1348 that is in fluid communication with the inlet air path 1330 will become smaller thereby resulting in less fragrance particles being dispensed into the debris cavity of the station dust cup 1210. In the closed position, the cross-sectional size of the entrance 1348 is no longer in fluid communication with the inlet air path 1330, air is substantially prevented from flowing through the odor control assembly 1300, and fragrance particles are substantially prevented from being disposed into the debris cavity of the station dust cup 1210.

FIG. 14C is a rear perspective view of an odor control assembly 1300 mounted in a station dust cup 1210 that shows the outlet air path 1430 between the exit 1350 of the puck cartridge 1308 and the station dust cup inlet port 1318. The outlet air path 1430 is formed by the outlet chamber 1324, which fluidly couples the exit 1350 to the station dust cup inlet port 1318.

FIG. 15 is a top cross-sectional view of an odor control assembly 1300 mounted in a station dust cup 1210 taken along line B-B of FIG. 11, showing the inlet air path 1530 as air enters through the one-way door 1320 and passes through the odor control assembly 1300 from entrance 1348, through the scent puck 1306, and out of the scent puck 1306 through the exit 1350. The air path 1530 then enters the station dust cup 1210 via the outlet chamber 1324.

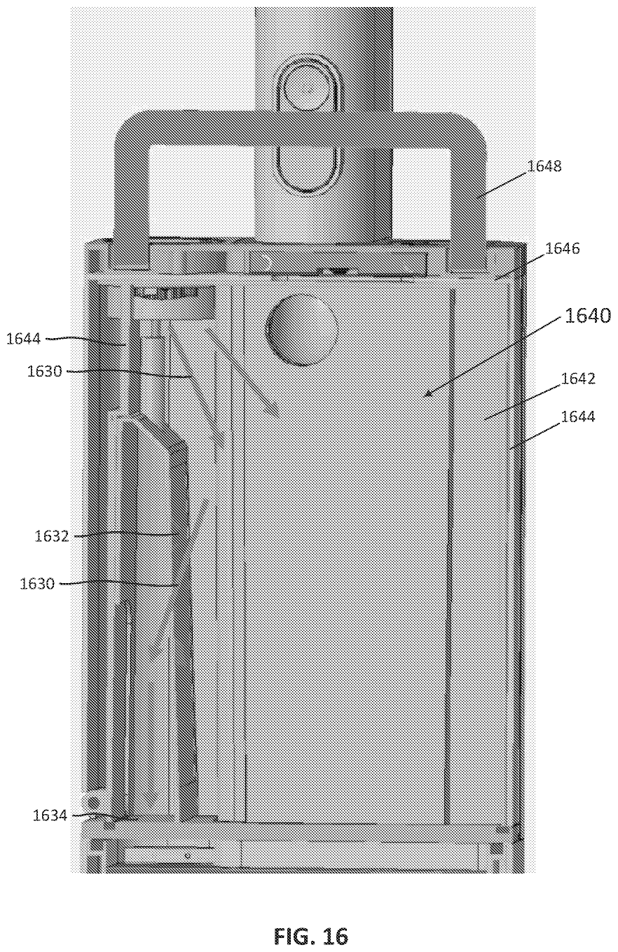

FIG. 16 is a front cross-sectional view of one embodiment of a station dust cup taken along line C-C of FIG. 11, showing the outlet air path 1630 into the station dust cup 1210. The suction created by the station suction motor 122 during the evacuation of debris from the cleaner dust cup 108 to draw air from the air path 1530 to draw fragrance particles from the scent puck 1306 into the debris cavity 1640 of the station dust cup 1210. The air drawn into the station dust cup 1210 by the station suction motor 122 ultimately exits the station dust cup 1220, e.g., via one or more station exhaust ports 1634. In the illustrated example, the station exhaust ports 1634 may be located proximate the bottom of the docking station 1200, though it should be appreciated that the station exhaust ports 1634 may be located anywhere on the station dust cup 1210. To prevent debris from being urged out of the debris cavity 1640 of the station dust cup 1210 through the station exhaust port 1634, the air passes through one or more output air filters 1632.

The station dust cup 1210 includes a body 1642 which at least partially defines the debris cavity 1640. The body may include, for example, one or more sidewalls 1644. The station dust cup 1210 may optionally include one or more covers 1646. The cover 1646 may be configured to generally seal with body 1642 and may be configured to transition to an empty position in which the user can remove debris from the debris cavity 1640. For example, the cover 1646 may be completely removable from the body 1642 or hingedly coupled to the body 1642. The body 1642 may optionally include one or more handles 1648.

The odor control assembly 1300 may be configured to be removably secured to the body 1642 and/or the cover 1646. In the illustrated example, the odor control assembly 1300 may be removably secured to a top sidewall 1644. The cover 1646 may be located proximate the bottom of the debris cavity 1640. The user may grasp the station dust cup 1210 by the handle, transition the cover 1646 to the empty position, and remove debris from the debris cavity 1640. By securing the odor control assembly 1300 to the body 1642, the user can empty the debris cavity 1640 without having to come in contact with the odor control assembly 1300. In addition, locating the odor control assembly 1300 on the top sidewall 1644 may generally prevent debris from coming into contact with the odor control assembly 1300, e.g., when the station suction motor 122 is turned off.

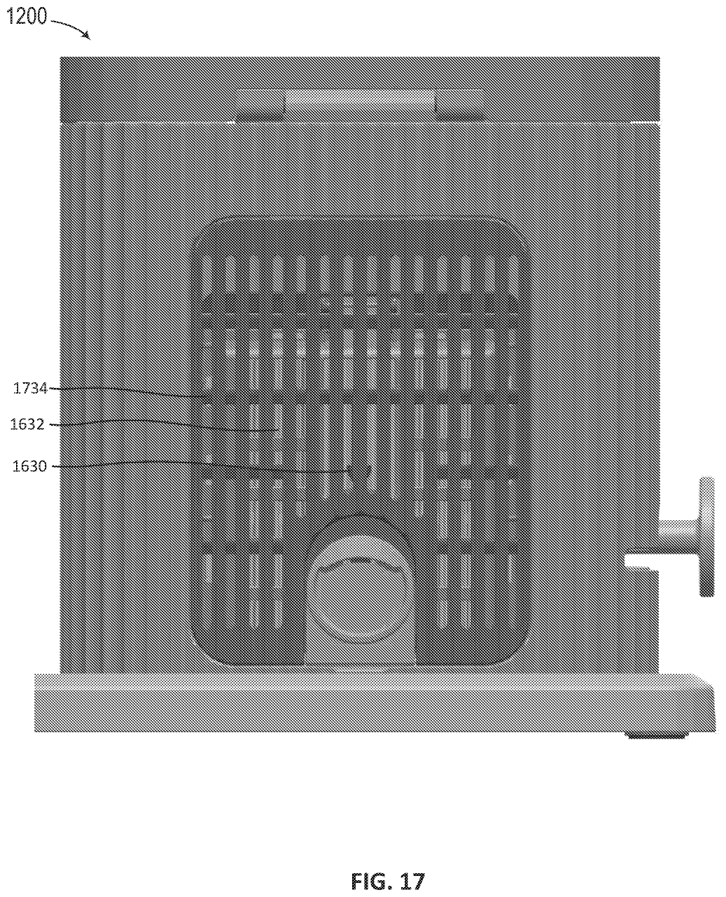

FIG. 17 is a side view of another embodiment of a docking station 1200 for the vacuum cleaner, showing a station exhaust port 1734 disposed on the side of the docking station 1200. In the embodiment of FIG. 17, air path 1630 still urges the output air through the output air filter 1632, and out through the station exhaust port 1734. It should be appreciated that the station exhaust port 1734 may be located anywhere on the docking station 1200.

While the docking station 1200 has been shown in combination with a hand-held vacuum cleaner 1100, it should be appreciated that the docking station 1200 may be used with any vacuum cleaner including, but not limited to, robotic vacuum cleaners. In some instances, the vacuum cleaner 1100 may have an odor control assembly 1300 fluidly coupled with the vacuum cleaner, in addition to, or in place of, the odor control assembly that is coupled with the station dust cup. In some instances, the odor control assembly for the vacuum cleaner and the odor control assembly for the docking station may be identical and interchangeable.

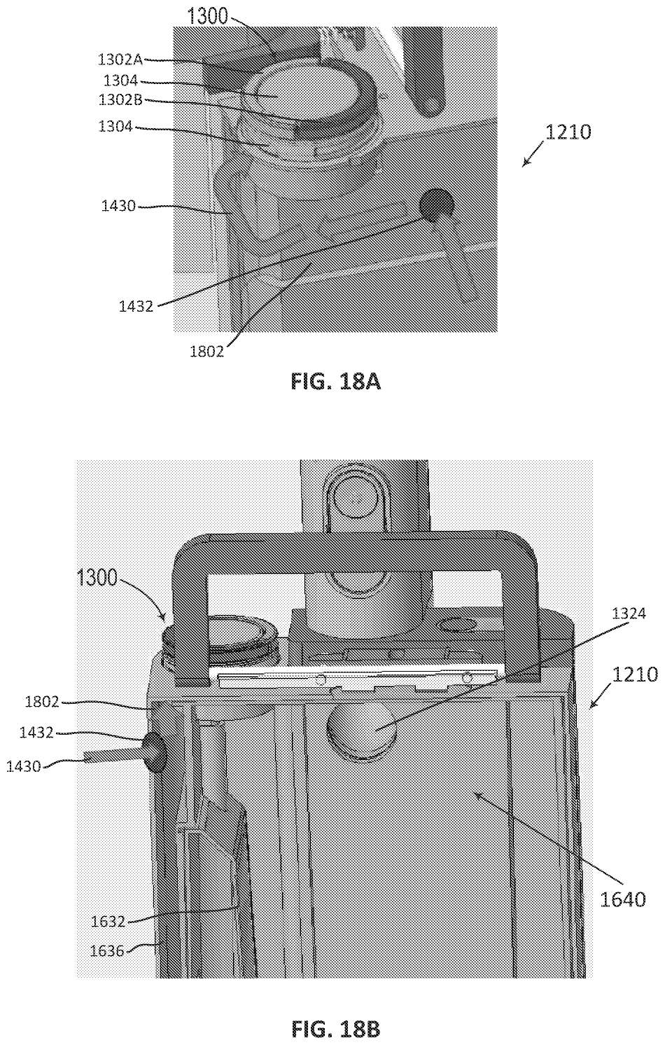

FIG. 18A is a perspective view of a station dust cup, showing an alternate bleed hole location for the inlet air for the odor control assembly. In some instances, the bleed hole 1432 may be disposed on the right side of the station dust cup 1210, as shown in FIG. 18A. In the instance shown in FIG. 18A, bleed hole 1432 is disposed on the right side of the station dust cup 1210 and allows air to enter through the bleed hole 1432 and travel through an air channel 1802 in the station dust cup 1210 to the entrance 1348 of the odor control assembly 1300.

FIG. 18B is a front cross-sectional view of a docking station 1200 taken along line C-C of FIG. 11, showing the alternate bleed hole 1432 location for the inlet air for the odor control assembly from FIG. 18A. The air channel 1802 is shown in this figure.

In other instances, the bleed hole 1432 may be located anywhere in the docking station 1200.

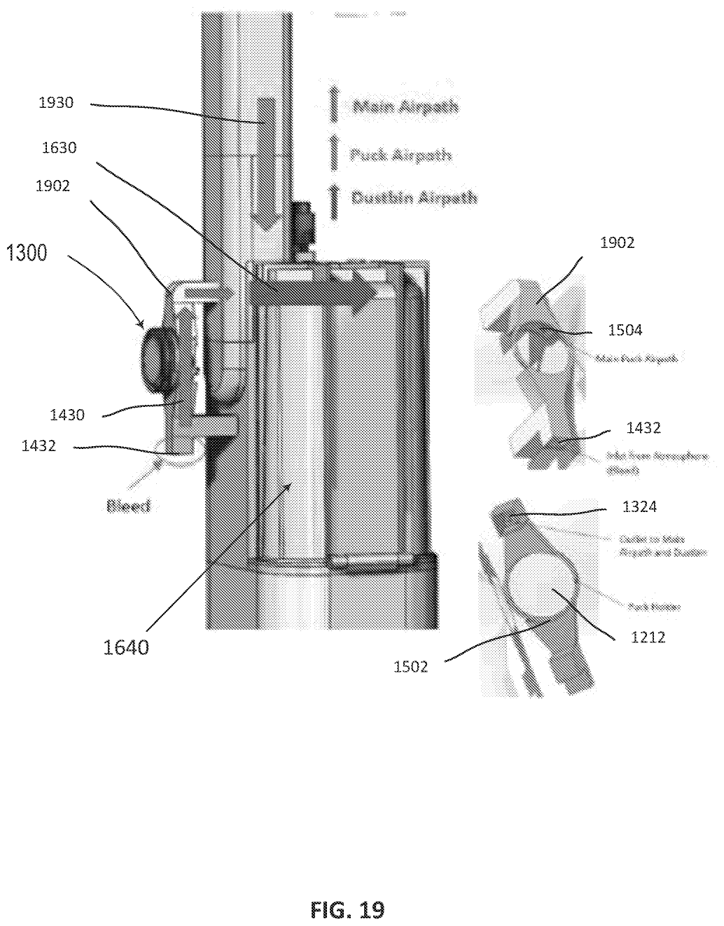

In some instances, odor control assembly 1300 may be located anywhere in the docking station 1200 that provides an air path to allow for the fragrance particles to be urged into the station dust cup 1210. FIG. 19 is a perspective view of a docking station 1200 for the vacuum cleaner 1100, showing an alternate location for the odor control assembly 1300. In FIG. 19, the odor control assembly 1300 may be disposed on the docking station 1200, rather than the station dust cup 1210 as described above. In this instance, the odor control assembly 1300 may be fluidly coupled to the up-duct 116, to use the suction of the main air path 1630, i.e., the air path used by the station suction motor to evacuate the cleaner dust cup 108 to draw air through the bleed hole 1432 and through the scent puck 1306, to output fragrance particles to the station dust cup 1210.

As shown in FIG. 19, odor control assembly 1300 is coupled to bracket 1902, which includes odor control cavity 1212. Bracket 1902 includes bleed hole 1432, which may be disposed at a first end of bracket 1902, and outlet chamber 1324, which may be disposed at a second end of bracket 1902, opposite from the first end of bracket 1902. Bracket 1902 may be mounted such that bleed hole 1432 is generally pointed in a downward direction when the docking station 1200 is in an upright position, i.e., ready to receive a vacuum cleaner 1100. This helps prevent debris from entering bleed hole 1432. Bracket 1902 also includes inlet port 1502 and outlet port 1504 for the odor control assembly 1300.

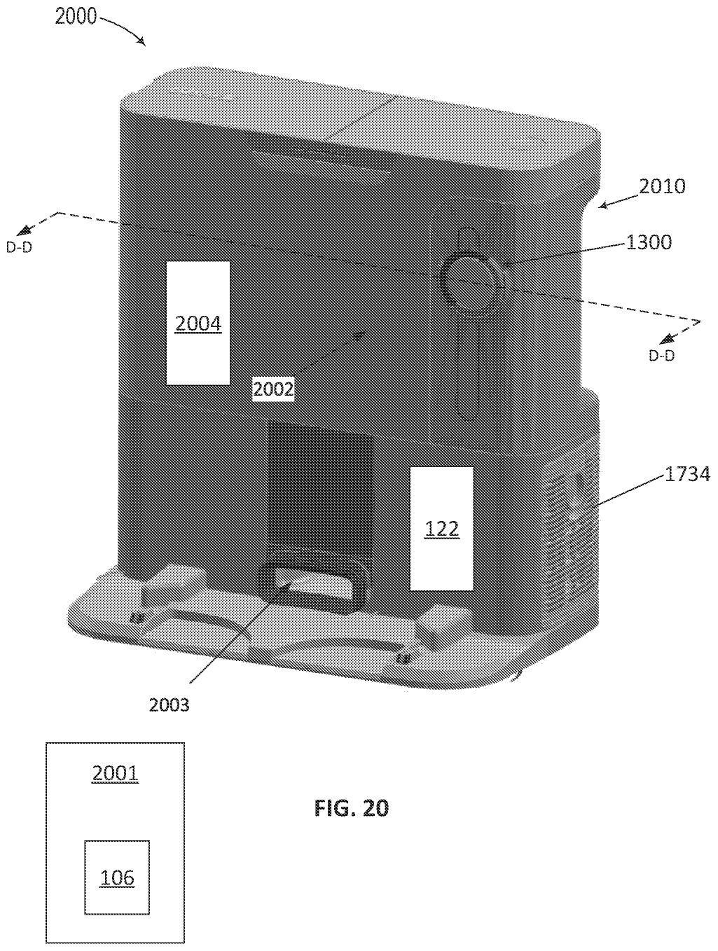

In some embodiments, the odor control assembly 1300 may be used in a docking station for a robot vacuum cleaner. FIG. 20 shows a front perspective view of a docking station 2000 for a robotic vacuum cleaner 2001 that incorporates one or more odor control assemblies 1300, which may be coupled to the robotic station dust cup 2010 that at least partially defines a debris cavity 2002. The docking station 2000 also includes a dirty air inlet 2003, one or more filters 2004, a station exhaust port 1734, and optionally a station suction motor 122. The dirty air inlet 2003 is configured to fluidly couple with the robotic vacuum cleaner 2001 in any manner known to those skilled in the art. The docking station 2000 and robotic vacuum cleaner 2001 are configured to transfer debris stored within the robotic vacuum cleaner 2001 to the robotic station dust cup 2010 in the docking station 2000. The debris may be transferred using one or more of the station suction motor 122 and/or the cleaner suction motor 106.

The dirty air inlet 2003 may be fluidly coupled to the robotic station dust cup 2010 (e.g., the debris cavity 2002). The one or more filters 2004 may be configured to remove at least some of the debris in the dirty air flow from the robotic vacuum cleaner 2001. The removed debris may be at least partially stored in the debris cavity 2002. The cleaned air may ultimately exit the robotic docking station 2000 via one or more station exhaust ports 1734.

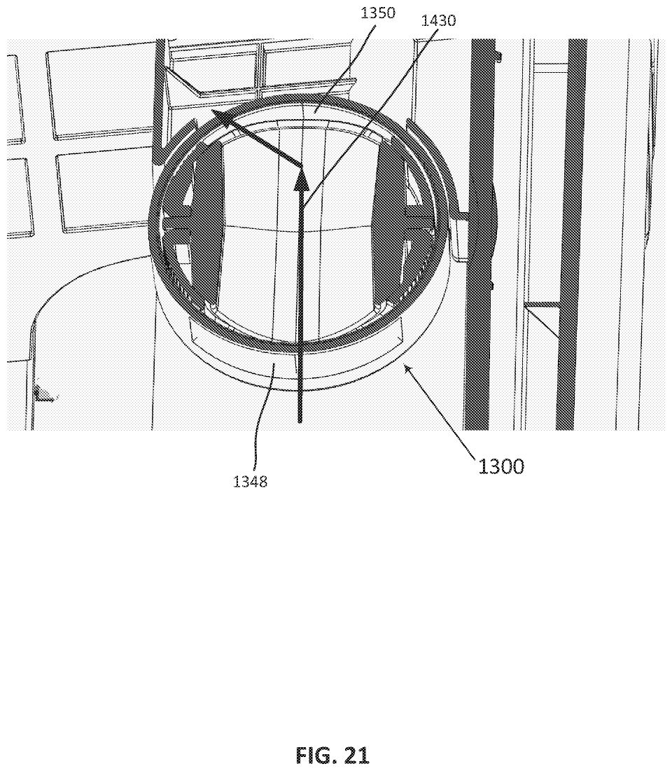

In one embodiment, the operation of the one or more odor control assemblies 1300 in the robotic docking station 2000 may include a bleed path (e.g., but not limited to, bleed hole and air path 1330) as generally described above for docking station 1200. For example, FIG. 21 is a front cross-sectional view of an odor control assembly in a docking station for a robotic vacuum cleaner taken along line D-D of FIG. 20, illustrating the inlet air path 1330. As in the docking station 1200, air is drawn in by suction created by the station suction motor 122 into entrance 1348, through the scent puck, and then exits the odor control assembly 1300 via the exit 1350. The outlet air from the odor control assembly 1300, with the fragrance particles, is then output into the robotic station dust cup 2010. In one embodiment, the air is drawn into entrance 1348 through a bleed hole (not shown) from the outside of the docking station.

Alternatively, the bleed path (e.g., bleed hole and air path 1330) may be eliminated. For example, the odor control assembly 1300 (e.g., the puck cartridge 1308 and/or scent puck 1306) may be at least partially exposed and/or disposed within the debris cavity 2002. Air may be drawn through the odor control assembly 1300 (e.g., the puck cartridge 1308 and/or scent puck 1306), for example, by suction from the station suction motor 122 and/or the cleaner suction motor 106. The odor control assembly 1300 may also dispense fragrance particles into the debris cavity 2002 without the use suction from the station suction motor 122 and/or the cleaner suction motor 106. For example, the odor control assembly 1300 may diffuse fragrance particles into the debris cavity 2002 by virtue of the odor control assembly 1300 being at least partially disposed within the debris cavity 2002.

According to one aspect of the disclosure there is thus provided a cleaning system, the system including: a docking station including: a station suction inlet configured to be fluidly coupled to a vacuum cleaner; a station dust cup configured to be removably fluidly coupled to the docking station, the station dust cup including a debris cavity; an odor control assembly fluidly coupled to the station dust cup; and a station suction motor configured to cause air to flow into the station suction inlet and through the station dust cup, wherein the station suction motor is configured to generate an airflow through the odor control assembly and into the debris cavity.

According to another aspect of the disclosure, there is thus provided a cleaning system including: a vacuum cleaner; a docking station, the vacuum cleaner configured to dock with the docking station, the docking station including: a station suction inlet configured to be fluidly coupled to the vacuum cleaner; a station dust cup configured to be removably fluidly coupled to the docking station, the station dust cup including a debris cavity; an odor control assembly fluidly coupled to the station dust cup; and a station suction motor configured to cause air to flow into the station suction inlet and through the station dust cup, wherein the station suction motor is configured to generate an airflow through the odor control assembly and into the debris cavity.

According to yet another aspect of the disclosure, there is provided a cleaning system including: a vacuum cleaner; a first odor control assembly fluidly coupled to the vacuum cleaner; a docking station, the vacuum cleaner configured to dock with the docking station, the docking station including: a station suction inlet configured to be fluidly coupled to the vacuum cleaner; a station dust cup configured to be removably fluidly coupled to the docking station, the station dust cup including a debris cavity; a second odor control assembly fluidly coupled to the station dust cup; and a station suction motor configured to cause air to flow into the station suction inlet and through the station dust cup, wherein the station suction motor is configured to generate an airflow through the second odor control assembly and into the debris cavity; and wherein the first odor control assembly and the second odor control assembly are interchangeable.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention, which is not to be limited except by the following claims.

What is claimed is:

1. A cleaning system comprising:
a docking station including:
a station suction inlet configured to be fluidly coupled to a vacuum cleaner;
a station dust cup configured to be removably fluidly coupled to the docking station, the station dust cup including a debris cavity;
an odor control assembly fluidly coupled to the station dust cup; and
a station suction motor configured to cause air to flow into the station suction inlet and through the station dust cup, wherein the station suction motor is configured to generate an airflow through the odor control assembly and into the debris cavity.

2. The cleaning system of claim 1, wherein the docking station further comprises a base configured to be removably secured to the station dust cup, the base including the station suction motor.

3. The cleaning system of claim 1, wherein the odor control assembly comprises:
a scent puck; and
a dial body configured to be removably secured to the station dust cup and configured to receive a scent puck, wherein the dial body at least partially defines a fragrance cavity configured to receive and generally enclose the scent puck.

4. The cleaning system of claim 3, wherein the odor control assembly further comprises:
one or more fragrance passageways, the one or more fragrance passageways configured to allow air to flow past the scent puck to transfer fragrance particles into the air to form fragranced air.

5. The cleaning system of claim 4, wherein the docking station further comprises:
a bleed hole disposed in an outer surface of the docking station, and configured to allow external air to be drawn into the docking station; and
an inlet air path to fluidly couple the bleed hole with the one or more fragrance passageways.

6. The cleaning system of claim 5, wherein the inlet air path further comprises a backflow preventor, wherein the backflow preventor is configured to seal off the odor control assembly from atmospheric air to substantially prevent air from escaping from the inlet air path.

7. The cleaning system of claim 6, wherein the backflow preventor includes a one-way valve.

8. The cleaning system of claim 4, wherein the dial body includes:
a puck cap; and
a puck cartridge removably coupled to the puck cap at least partially forming the fragrance cavity and the one or more fragrance passageways, the puck cap including an entrance and an exit to the one or more fragrance passageways, wherein atmospheric air flows through the entrance, across the scent puck, and out of the exit.

9. The cleaning system of claim 8, wherein the puck cap further comprises:
a fixed ring secured to the puck cap; and
one or more rotatable sections that function as a handle to aid in insertion and removal of the odor control assembly into the station dust cup.

10. The cleaning system of claim 8, wherein the puck cartridge further comprises:
a base; and
one or more sidewalls extending upwardly from the base, wherein:
the one or more sidewalls defining a puck chamber configured to receive the scent puck;
the one or more sidewalls at least partially defining the entrance and the exit to the one or more fragrance passageways;
the entrance and the exit to the one or more fragrance passageways being generally aligned 180 degrees opposite each other; and
the one or more sidewalls include one or more puck alignment features to align the one or more fragrance passageways with the entrance and the exit.

11. The cleaning system of claim 10, wherein the odor control assembly is configured to rotate within the station dust cup between a first position to a second position to adjust at least one of a cross-sectional size of the entrance or the exit to the one or more fragrance passageways to cover or expose the one or more fragrance passageways thereby regulating an amount of air drawn through the one or more scent pucks.

12. The cleaning system of claim 8, wherein the station dust cup includes an outlet air path formed by an outlet chamber between the exit from the one or more fragrance passageways and a station dust cup inlet port, wherein the outlet air path draws the fragranced air from the odor control assembly into the station dust cup.

13. The cleaning system of claim 1, further comprising an odor control cavity at least partially disposed in a top surface of the station dust cup, the odor control cavity configured to at least partially receive the odor control assembly.

14. A cleaning system comprising:
a vacuum cleaner;
a docking station, the vacuum cleaner configured to dock with the docking station, the docking station including:
a station suction inlet configured to be fluidly coupled to the vacuum cleaner;
a station dust cup configured to be removably fluidly coupled to the docking station, the station dust cup including a debris cavity;
an odor control assembly fluidly coupled to the station dust cup; and
a station suction motor configured to cause air to flow into the station suction inlet and through the station dust cup, wherein the station suction motor is configured to generate an airflow through the odor control assembly and into the debris cavity.

15. The cleaning system of claim 14, wherein the docking station further comprises a base configured to be removably secured to the station dust cup, the base including the station suction motor.

16. The cleaning system of claim 14, wherein the odor control assembly comprises:
a scent puck; and
a dial body configured to be removably secured to the station dust cup and configured to receive a scent puck, wherein the dial body at least partially defines a fragrance cavity configured to receive and generally enclose scent puck.

17. The cleaning system of claim 16, wherein the odor control assembly further comprises:
one or more fragrance passageways, the one or more fragrance passageways configured to allow air to flow past the scent puck to transfer fragrance particles into the air to form a fragranced air that flows into the debris cavity.

18. The cleaning system of claim 14, wherein the vacuum cleaner is a handheld vacuum cleaner.

19. The cleaning system of claim 14, wherein the vacuum cleaner is a robotic vacuum cleaner.

20. A cleaning system comprising:
a vacuum cleaner;
a first odor control assembly fluidly coupled to the vacuum cleaner;
a docking station, the vacuum cleaner configured to dock with the docking station, the docking station including:
a station suction inlet configured to be fluidly coupled to the vacuum cleaner;
a station dust cup configured to be removably fluidly coupled to the docking station, the station dust cup including a debris cavity;
a second odor control assembly fluidly coupled to the station dust cup; and
a station suction motor configured to cause air to flow from the vacuum cleaner into the station suction inlet and through the station dust cup, wherein the station suction motor is configured to generate an airflow through the second odor control assembly and into the debris cavity.
